(19) United States
(12) Patent Application Publication
(10) Pub. No.: US 2010/0316672 A1
(43) Pub. Date: Dec. 16, 2010

(54) VACCINE

(76) Inventors: Barton F. Haynes, Durham, NC (US); Hua-xin Liad, Durham, NC (US); Ben-jiang Ma, Durham, NC (US); Joseph Sodroski, Boston, MA (US)

Correspondence Address: NIXON & VANDERHYE, PC, 901 NORTH GLEBE ROAD, 11TH FLOOR, ARLINGTON, VA, 22203, US

(21) Appl. No.: 12/450,774

(22) PCT Filed: Apr. 10, 2008
(86) PCT No.: PCT/US2008/004579

§ 371 Date: Aug. 23, 2010

Related U.S. Application Data
(60) Provisional application No. 60/907,719, filed on Apr. 13, 2007.

(51) Int. Cl. A61K 39/21; C07H 21/00; A61K 31/7088; C07K 14/16; A61P 37/04; A61P 31/18

(52) U.S. Cl. 424/208.1; 536/23.72; 514/44 R; 530/350

(57) ABSTRACT

The present invention relates, in general, to human immunodeficiency virus (HIV) and, in particular, to HIV-I envelope (Env) immunogens.

This application claims priority from U.S. Provisional Application No. 60/907,719, filed Apr. 13, 2007, the entire content of which is incorporated herein by reference.

This invention was made with government support under Grant No. AI067854 awarded by the National Institutes of Health. The government has certain rights in the invention.

### TECHNICAL FIELD

The present invention relates, in general, to human immunodeficiency virus (HIV) and, in particular, to HIV-1 envelope (Env) immunogens.

### BACKGROUND

It has been hypothesized that some of the quantitative and qualitative abnormalities in immune responses in HIV-1 infection may be due to the presence of immunosuppressive activity of gp160 mediated by Env superantigen (SA) activity (Karray et al, Proc. Natl. Acad. Sci. USA 94(4):1356-1360 (1997)) or by immunosuppressive effects of gp120 binding to CD4 on T cells, macrophages or DCs (Pantaleo et al, N. Engl. J. Med. 328(5):327-335 (1993), Vingerhoets et al, Clin. Exp. Immunol. 111(1):12-19 (1998)). The present invention results, at least in part, from studies designed to test this hypothesis. These studies included the production of HIV-1 Envs that express epitopes to which broadly neutralizing antibodies can bind and the mutation of such Envs such that they have no superantigen activity and/or they cannot bind immune cell CD4 in an immunosuppressive manner. The present invention relates to such mutated envelopes and to methods of inducing an immune response using same.

### SUMMARY OF THE INVENTION

The present invention relates generally to HIV and, more specifically, to immunogenic compositions and methods of inducing an immune response against HIV using same.

Objects and advantages of the present invention will be clear from the description that follows.

### BRIEF DESCRIPTION OF THE DRAWINGS

FIG. 1. Schematic structure of HIV-1 JRFL Env and mutant JRFL Envs with mutation at CD4 binding site and superantigen motif.

FIG. 2. Western blot analysis and ELISA assay of HIV-1 JRFL mutant gp140 Envs.

FIG. 3. Surface plasma resonance analysis of HIV-1 JRFL mutant gp140 Envs.

### DETAILED DESCRIPTION OF THE INVENTION

The invention is exemplified below with respect to HIV-1 envelope (Env) which contains various antigenic epitopes such as CD4 binding site, variable loops, MPER 4E10 and 2F5 neutralizing epitopes as well as other neutralizing epitopes. HIV-1 Envs used as immunogens to date induce antibodies that only neutralize selected HIV-I primary isolates. To test the hypothesis that one reason that broadly neutralizing antibodies cannot be made is due to SAg activity and or CD4 binding immunosuppressive activity, a strategy has been developed for: 1) removing the SAg-binding motif on HIV-1 Env gp140CF oligomer, and 2) disrupting the CD4 binding site of HIV Env oligomer.

HIV-1 subtype B primary isolate JRFL is a tier 2 virus that is a relatively difficult isolate to neutralize, yet has both MPER 4E10 and 2F5 gp41 broadly neutralizing epitopes expressed well on this oligomer (Liao et al, Virology 353:268-282 (2006)). A JRFL gp140 WT immunogen induced antibodies that neutralized only a select few subtype β isolates but did not neutralize its autologous JRFL isolate (Liao et al, Virology 353:268-282 (2006)). Experiments were performed using JRFL Env 140 oligomer as a prototype (see Example below).

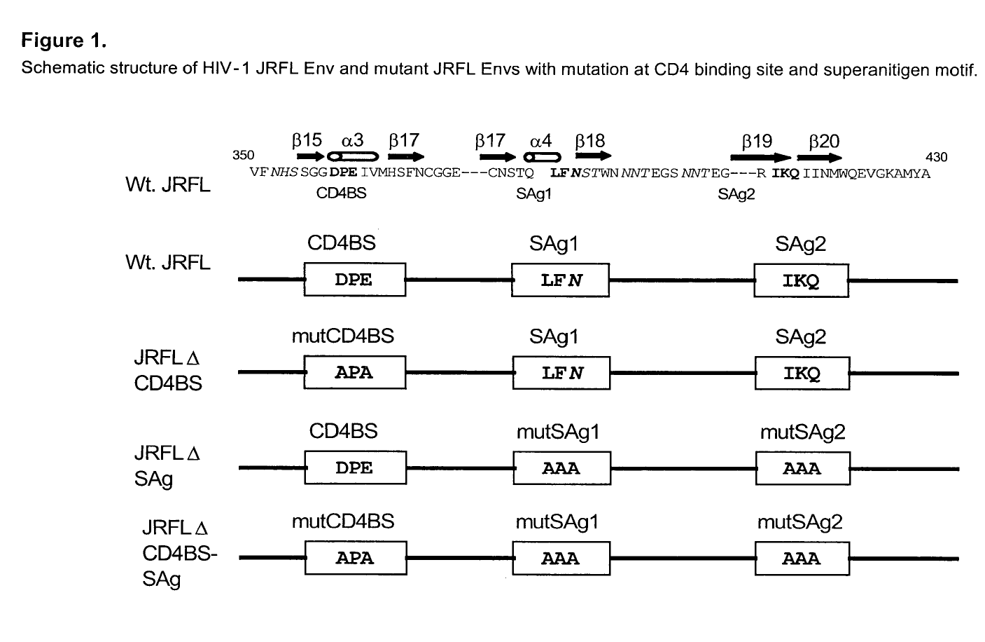

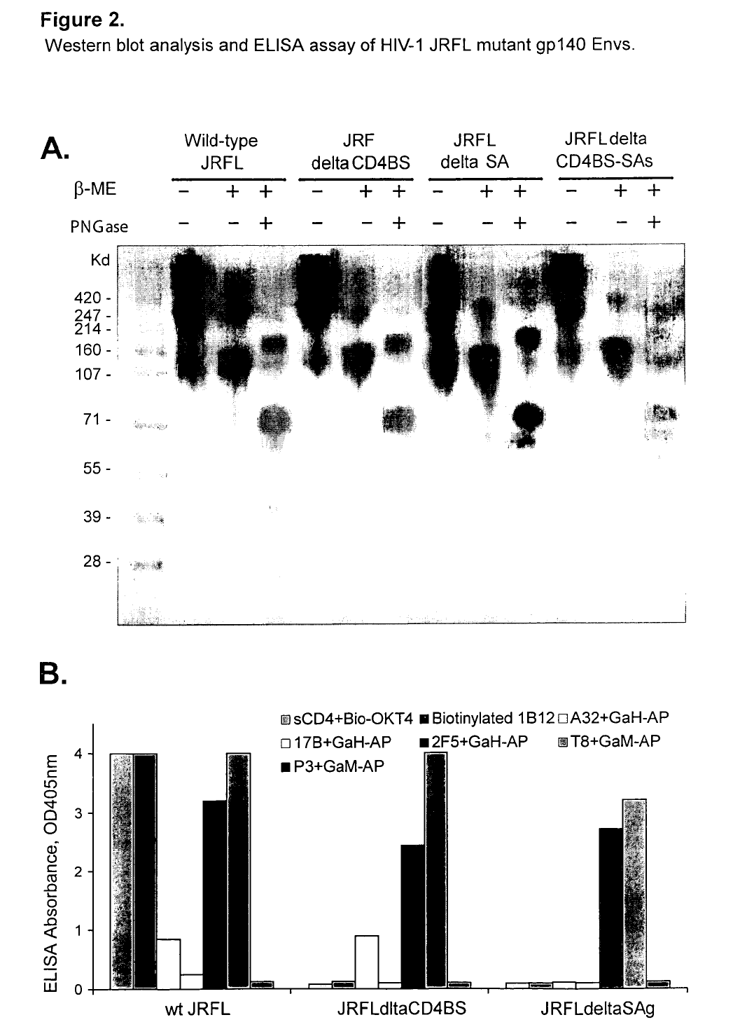

Three mutant JRFL gp140 expression constructs were designed and generated (FIG. 1) using pcDNA3.1 plasmid (Invitrogen, Carlsbad, Calif.). Stably transfected 293T cell lines have been established to produce recombinant JRFL gp140 with CD4 binding site mutated (JRFLΔCD4BS), JRFL gp140 with deletions of SA binding motif (JRFLΔSAg) and JRFL gp140 with both CD4 binding site and superantigen motif mutated (JRFLΔCD4BS-SAg). Recombinant proteins of all three were expressed and purified from the supernatants of the stably transfected 293T cell lines by lectin columns (FIG. 2A). Western blot analysis using HIV-1 gp120 MAb T8, JRFL mutant Envs with or without deglycosylation with PNGase digestion showed no differences in apparent migration patterns in SDS-PAGE under reducing or non-reducing conditions in comparison with the wild-type JRFL Env (FIG. 2A). ELISA assays demonstrated that mutation either at the CD4 binding site or at the SA motif maintained the ability to bind gp120 MAb T8 and MPER MAbs 2F5 and 4E10, while abrogated the ability of these mutant Envs to bind CD4 and CD4 binding site MAb, 1B12 (FIG. 2B). JRFLASAg mutant Env also lost the ability to bind to CD4i MAb A32 (FIG. 2B).

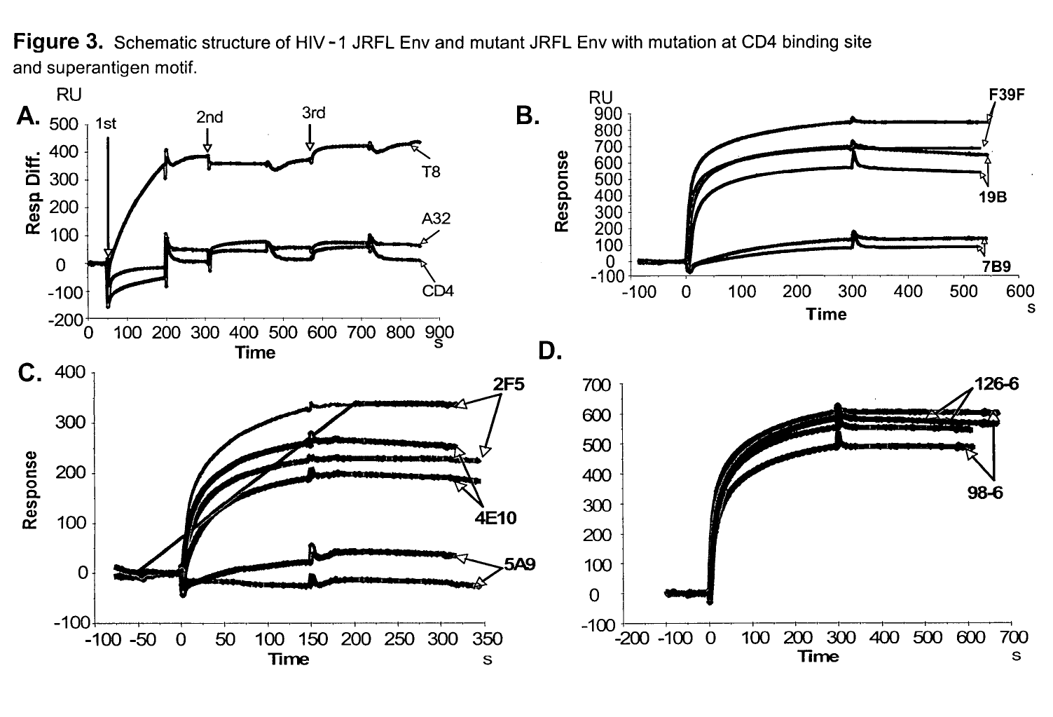

Functional and antigenic epitopes on JRFL Env mutants were further characterized by surface plasma resonance analysis (FIG. 3). It has been found that JRFLΔCD4BS Env strongly bound HIV gp120 MAb T8 and bound MAb A32 at low levels (FIG. 3A), while no constitutive binding of MAb 17B, or anti-gp41 MAb 7B2 binding to JRFLΔCD4BS Env was observed. Substitution of amino acids DPE with APA at one of CD4 binding touch points completely abolished the ability of JRFLΔCD4BS Env to bind CD4 (FIG. 3A). Various anti-HIV-1 V3 antibodies also bound to both JRFL gp140 Env (FIG. 3B, solid lines) as well as to JRFLΔCD4BS gp140 Env (FIG. 3B, broken lines). HIV-1 MPER neutralizing epitopes were preserved as HIV-1 MPER mAbs 2F5 and 4E10 bound in comparable levels to both JRFL gp140 (FIG. 3C, solid line) and JRFLΔCD4BS gp140 (FIG. 3C, broken line). However JRFLΔCD4BS gp140 did not bind to the non-neutralizing murine MPER MAb 5A9, which bound to JRFL gp140 with low avidity, while strong binding of human cluster II MAb 98-6 and 126-6 to both JRFL gp140 (FIG. 3D, solid lines) and JRFLΔCD4BS gp140 (FIG. 3D, broken lines) was observed. A study of the functional and immunogenic properties of JRFL Env with mutations at both CD4 binding site and SA motif are in progress.

The immunogen of one aspect of the invention comprises an envelope either in soluble form or anchored, for example, in cell vesicles or in liposomes containing translipid bilayer envelope. To make a more native envelope, sequences can be configured in lipid bilayers for native trimeric envelope formation. Alternatively, the invention, in the form of gp160, can be used as an immunogen.

The immunogen of the invention can be formulated with a pharmaceutically acceptable carrier and/or adjuvant (such as alum or oCpG) using techniques well known in the art. Suitable routes of administration of the present immunogen include systemic (e.g., intramuscular or subcutaneous). Alternative routes can be used when an immune response is sought in a mucosal immune system (e.g., intranasal).

The immunogens of the invention can be chemically synthesized or synthesized using well-known recombinant DNA techniques. Nucleic acids encoding the immunogens of the invention can be used as components of, for example, a DNA vaccine wherein the encoding sequence is administered as naked DNA or, for example, a minigene encoding the immunogen can be present in a viral vector. The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. Expression of the immunogen of the invention can be induced in a patient's own cells, by introduction into those cells of nucleic acids that encode the immunogen, preferably using codons and promoters that optimize expression in human cells. Examples of methods of making and using DNA vaccines are disclosed in U.S. Pat. Nos. 5,580,859, 5,589,466, and 5,703,055.

The invention further relates to a composition comprising an immunologically effective amount of the immunogen of this invention, or nucleic acid sequence encoding same, in a pharmaceutically acceptable delivery system. The compositions can be used for prevention and/or treatment of immunodeficiency virus infection. The compositions of the invention can be formulated using adjuvants, emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal administration). The present compositions can be administered by any convenient route including subcutaneous, intranasal, oral, intramuscular, or other parenteral or enteral route. The immunogens can be administered as a single dose or multiple doses. Optimum immunization schedules can be readily determined by the ordinarily skilled artisan and can vary with the patient, the composition and the effect sought.

The invention contemplates the direct use of both the immunogen of the invention and/or nucleic acids encoding same and/or the immunogen expressed as minigenes in the vectors indicated above. For example, a minigene encoding the immunogen can be used as a prime and/or boost.

Certain aspects of the invention are described in greater detail in the non-limiting Example that follows. (See also U.S. application Ser. No. 10/572,638.)

### Example 1

### Cloning of JRFL Env gn140CF with Mutation at the CD4 Binding Site

The amino acid sequence at position 358 to 360 (DPE) was one of touch points when HIV-1 Env binds to CD4 (Kwong et al, Nature 398:648-659 (1998)). To mutate CD4 binding site on JRFL Env, 2 pairs of the mutagenic primers were designed and synthesized for use in PCR (Table 1) to introduce mutations in gene sequence by changing the coding sequence for DPE to the coding sequence for APA by PCR. HIV-1 JRFL gp140CF gene construct (Liao et al, Virology 353:268-282 (2006)) was used as template in PCR amplification to produce JRFL Env mutant genes. Two sets of the first round PCR were performed to introduce the site-specific mutations and generate the first half and the second half of the JRFL140 DNA fragments. The first half JRFL 140 DNA fragment was amplified by using the primer pair of the forward primer (JRFL-F1) and reverse primer (JRFL-mut1165). The second half JRFL 140 DNA fragment was amplified by using the primer pair of the forward primer (JRFL-mutF 1142) and reverse primer (JRFL-R1978) (Table 1). The amplified two JRFL DNA fragments from these 2 sets of PCR (10 ng of each) were used as templates for the second round of PCR to produce the full-length JRFL 140 gene using the primer pair of JRFL-F1 and JRFL-R1978. All PCRs were carried out in total volume of 50:1 using 1 unit of AccuPrime Taq Polymerase High Fidelity (Invitrogen; Carlsbad, Calif.), and 50 pmol of each primer. The PCR thermocycling conditions were as follows: one cycle at 94° C. for 1 min; 25 cycles of a denaturing step at 94° C. for 30 sec, an annealing step at 55° C. for 30 sec, an extension step at 68° C. for 2 min; and one cycle of an additional extension at 68° C. for 5 min. The resulting full-length JRFL 140 DNA fragment was purified with PCR purification column (Qiagen) and enzymatic digestion with restriction enzyme SalI and BamHI, and then cloned to expression vector pcDNA3.1 (−)/Hygro (Invitrogen Co, CA) via Xba I and BamH I site. The resulting DNA clones of JRFL with the CD4 BS mutated (pJRFLΔCD4 BS) were validated by DNA sequencing of full-length of the gene construct.

List of Table 1 PCR primers used in PCR.

| Primer Name | Primer Sequence (5′ to 3′) | Purpose |
|---|---|---|
| JRFL-F1 | TTCAGCTAGC GTCGACGACCATGCCCATGGGGTCTCTGC | |
| JRFL-R1978 | GTGTGTGGATCCGGTACCCTACCACAGCCACTTGGTGATGTC | |
| JRFL-mutF1142 | GGTGGTGCCCCTGCCATTGTGATGCACAGCTTCAACTGTGGTGGTGAGTTCTTC | CD4 BS mutant |
| JRFL-mutR1165 | CATCACAATGGCAGGGGCACCACCAGAGCTGTGATTGAACAC | |
| JRFL-mutF1128 | CAGCACCCAGGCGGCCGCCAGCACCTGGAACAACAACACTGAGGGCAGCAACAACACTGA | Super antigen |
| | GGCAACACCATCACCCTGCCTTGCAGGGCCGCGGCGATCATCAACATGTGGCAG | mutant |
| JRFL-mutR1237 | CATGTTGATGATCGCCGCGGCCCTGCAAGGCAGGGTGATGGTGTTGCCCTCAGTGTTGTT | Super antigen |
| | CTGCCCTCAGTGTTGTTGTTCCAGGTGCTGGCGGCCGCCTGGGTGCTGTTGCAG | mutant |

Cloning of JRFL Env gp140CF with Mutation at the superantigen (SAg) motif. The superantigen-binding site is formed by protein sequences from two regions of HIV-1 gp120. The core motif is a discontinuous epitope spanning the V4 variable region and the amino-terminal region flanking the C4 constant domain. The amino acid sequence at position 358 to 360 (APA) was one of touch points when HIV-1 Env binds to CD4 (Karray et al, Proc. Natl. Acad. Sci. USA 94(4):1356-1360 (1997)). To disrupt the superantigen binding site, a primer pair (Table 1, JRFL-F1128 and JRFL-R1237) was designed to change the coding sequence for LFN at the SAg1 region to the coding sequence for AAA and change the coding sequence for IKQ at the SAg2 region to the coding sequence for AAA (FIG. 1). HIV-1 JRFL gp140CF gene construct (Liao et al, Virology 353:268-282 (2006)) was used as template in PCR amplification to produce JRFL Env mutant genes. Two sets of the first round PCR were performed to introduce the site-specific mutations and generate the first half and the second half of the JRFL140 DNA fragments. The first half JRFL 140 DNA fragment was amplified by using the primer pair of the forward primer (JRFL-F1) and reverse primer (JRFL-mut-R1237). The second half JRFL 140 DNA fragment was amplified by using the primer pair of the forward primer (JRFL-mut F1128) and reverse primer (JRFL-R1978) (Table 1). The amplified two JRFL DNA fragments from these 2 sets of PCR (10 ng of each) were used as templates for the second round of PCR to produce the full-length JRFL 140 gene using the primer pair of JRFL-F1 and JRFL-R1978. All PCRs were carried out in total volume of 50 μl using 1 unit of ΔccuPrime Taq Polymerase High Fidelity (Invitrogen; Carlsbad, Calif.), and 50 μmol of each primer. The PCR thermocycling conditions were as follows: one cycle at 94° C. for 1 min; 25 cycles of a denaturing step at 94° C. for 30 sec, an annealing step at 55° C. for 30 sec, an extension step at 68° C. for 2 min; and one cycle of an additional extension at 68° C. for 5 min. The resulting full-length JRFL 140 DNA fragment was purified with PCR purification column (Qiagen) and enzymatic digestion with restriction enzyme SalI and BamHI, and then cloned to expression vector pcDNA3.1 (−)/Hygro (Invitrogen Co, CA) via Xba I and BamH I site. The resulting DNA clones of JRFL with the superantigen binding region mutated (pJRFLtSAg) were validated by DNA sequencing of full-length of the gene construct.

Cloning of JRFL Env gp140CF with mutations at both CD4BS and the superantigen (SAg) motif. To disrupt both CD4BS and the superantigen binding site, HIV-1 JRFLiCD4SAg DNA construct was used as template in PCR amplification to produce JRFL Env mutant genes. Two sets of the first round PCR were performed to introduce the site-specific mutations and generate the first half and the second half of the JRFL 140 DNA fragments. The first half JRFL 140 DNA fragment was amplified by using the primer pair of the forward primer (JRFL-F1) and reverse primer (JRFL-mut1237). The second half JRFL 140 DNA fragment was amplified by using the primer pair of the forward primer (JRFL-mutF1128) and reverse primer (JRFL-R1978) (Table 1). The amplified two JRFL DNA fragments from these 2 sets of PCR (10 ng of each) were used as templates for the second round of PCR to produce the full-length JRFL 140 gene using the primer pair of JRFL-F1 and JRFL-R1978. All PCRs were carried out in total volume of 50 μl using 1 unit of ΔccuPrime Taq Polymerase High Fidelity (Invitrogen; Carlsbad, Calif.), and 50 μmol of each primer. The PCR thermocycling conditions were as follows: one cycle at 94° C. for 1 min; 25 cycles of a denaturing step at 94° C. for 30 sec, an annealing step at 55° C. for 30 sec, an extension step at 68° C. for 2 min; and one cycle of an additional extension at 68° C. for 5 min. The resulting full-length JRFL 140 DNA fragment were purified with PCR purification column (Qiagen) and enzymatic digestion with restriction enzyme SalI and BamHI, and then cloned to expression vector pcDNA3.1 (−)/Hygro (Invitrogen Co, CA) via Xba I and BamH I site. The resulting DNA clones of JRFL with the CD4 BS mutated (pJRFLΔCD4BS-SAg) were validated by DNA sequencing of full-length of the gene construct.

Generation of Stable Cell Lines and Expression: A human cell line 293T was used for establishing a stably transfected cell lines for expressing mutant JRFL Envs. 293T cells in tissue culture plates were transfected with either pJRFLΔCD4BS, pJRFLΔCDBS-SAg, or pJRFLΔCD4BS-SAg plasmid. Stabley transfected 293T cell clones that were resistant to hygromycin were selected in to culture medium containing 20% fetal bovine serum and hygromycin (200 μg/ml). Hygromycin-resistant clones were further cloned by the limiting dilution to select single colonies under hygromycin pressure (200 μg/ml). The individual cell lines that express JRFLLCD4BS, JRFLΔCDBS-SAg, or JRFLΔCD4BS-SAg gene constructs were confirmed to being correct by DNA sequencing.

All documents and other information sources cited above are hereby incorporated in their entirety by reference.

1. An immunogen comprising an HIV-1 envelope (Env) protein comprising a CD4 binding site mutation or a superantigen (SAg) binding motif mutation.

2. The immunogen according to claim 1 wherein said Env protein comprises gp140.

3. The immunogen according to claim 1 wherein said immunogen comprises a CD4 binding site mutation and a SAg binding motif mutation.

4. The immunogen according to claim 1 wherein said Env protein comprises said CD4 binding site mutation and wherein said immunogen does not bind CD4.

5. The immunogen according to claim 2 wherein said mutation is at one or more of the amino acids at positions 358 to 360.

6. The immunogen according to claim 5 wherein the mutation results in amino acid sequence APA at positions 358 to 360.

7. The immunogen according to claim 1 wherein said Env protein comprises said SAg binding motif mutation.

8. The immunogen according to claim 7 wherein the sequence LFN at the SAg1 motif is mutated to AAA and the sequence IKQ at the SAg2 motif is mutated to AAA.

9. The immunogen according to claim 1 wherein monoclonal antibodies 2F5 or 4E10 bind said immunogen.

10. A composition comprising said immunogen according to claim 1 and a carrier.

11. A nucleic acid construct comprising a sequence encoding the immunogen according to claim 1.

12. A composition comprising the nucleic acid according to claim 11 and a carrier.

13. A method of inducing an immune response in a mammal comprising administering to said mammal an amount of the immunogen according to claim 1 sufficient to effect said induction.

14. A method of inducing an immune response in a mammal comprising administering to said mammal said nucleic acid according to claim 11 under conditions such that said sequence is expressed, said immunogen is produced and said induction is effected.

15. The method according to claim 13 wherein said mammal is a human.
